(19) United States
(12) Patent Application Publication
(10) Pub. No.: US 2026/0232674 A1
(43) Pub. Date: Aug. 13, 2026

(54) METHODS OF TREATING HIDRADENITIS SUPPURATIVA

(71) Applicant: Alumis Inc., South San Francisco, CA (US)

(72) Inventors: Mera Tilley, South San Francisco, CA (US); Joshua Hoffman, South San Francisco, CA (US); Claire Langrish, South San Francisco, CA (US)

(21) Appl. No.: 19/157,101

(22) PCT Filed: Feb. 16, 2024
(86) PCT No.: PCT/US2024/016104

§ 371 Date: Aug. 15, 2025

Related U.S. Application Data
(60) Provisional application No. 63/485,397, filed on Feb. 16, 2023.

(51) Int. Cl. A61K 31/519; A61K 31/4439; A61K 31/4985; A61K 31/501; A61K 31/506; A61K 45/06; A61P 17/00

(52) U.S. Cl. CPC A61K 31/519; A61K 31/4439; A61K 31/4985; A61K 31/501; A61K 31/506; A61K 45/06; A61P 17/00

(57) ABSTRACT

The disclosure is directed, in part, to methods of treating a TYK2-mediated disorder, e.g., hidradenitis suppurativa (HS). For example, the disclosure provides a method of treating a patient suffering from moderate or severe hidradenitis suppurativa comprising administering to the patient an effective amount of a TYK2 inhibitor.

### CROSS REFERENCE TO RELATED APPLICATIONS

This application claims the benefit of, and priority to, U.S. Provisional Patent Application No. 63/485,397, filed on Feb. 16, 2023; the content of which is hereby incorporated by reference herein in its entirety.

### BACKGROUND

Hidradenitis suppurativa (HS) is a chronic inflammatory follicular occlusive disease and is characterized by inflammatory nodules, abscesses, scarring, and fibrosis. The occlusion of hair follicles in intertriginous skin areas can lead to follicular dilation and cyst formation, which results in rupture of the hair follicle. The introduction of follicular contents to the surrounding dermis induces an inflammatory response and formation of, e.g., abscesses, fibrosis, and scars.

Pro-inflammatory (e.g., IL-12, IL-17, IL-23) and anti-inflammatory (e.g., IL-10) cytokines are found to be increased in HS lesional and perilesional skin. Histologic findings in HS lesions suggest that the increase in cytokines results in neutrophil migration to the skin and formation of neutrophil extracellular traps. The enhanced neutrophil extracellular trap response contributes to increase in inflammation and activation of other immune cells including skin dendritic cells, thereby leading to increased type I interferon (IFN) response in HS lesions. The pathogenic effect of the IL-23/IL-17 axis is mediated by the janus kinase/signal transducers and activators of transcription (JAK/STAT) pathway.

TYK2 is a non-receptor tyrosine kinase member of the Janus kinase (JAKs) family of protein kinases and are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include, e.g., central cytokines IL-12 and IL-23 and interferons (e.g., IFN-α, IFN-β).

Inhibition of TYK2 significantly reduces both IL-23 and IL-22-induced dermatitis. Adalimumab, a TNFα inhibitor, is the only Food and Drug Administration (FDA)-approved biological drug for the treatment of moderate to severe HS with most other biologics/cytokine selective inhibitors (eg, IL-12/23 and IL-17) still in their early phases of development. Clinical efficacy of all novel treatments reported so far is modest.

Thus, HS remains a challenging disease to treat, and a significant unmet need exists for an effective treatment. Accordingly, given the apparent correlations between TYK2 and the immune pathways involved in pathogenesis of HS, there is a need to provide methods for inhibiting the activity of TYK2 to treat patients with moderate to severe HS.

### SUMMARY

The disclosure is directed, in part, to methods of treating a TYK2-mediated disorder, e.g., hidradenitis suppurativa (HS). For example, provided herein is a method of treating a patient suffering from moderate or severe hidradenitis suppurativa, and in need of treatment, comprising administering to the patient an effective amount of a TYK2 inhibitor.

Also disclosed herein is a method of treating moderate or severe hidradenitis suppurativa in a patient having elevated Type I IFN-induced sialic acid-binding immunoglobulin-like lectin1 (SIGLEC1) expression, and in need of treatment, comprising administering to the patient an effective amount of a TYK2 inhibitor, and thereby upon administration reducing the expression of SIGLEC1 in the patient.

Further described herein, for example, is a method of treating a patient having elevated sialic acid-binding immunoglobulin-like lectin1 (SIGLEC1) expression and suffering from moderate to severe hidradenitis suppurativa, comprising administering to the patient an effective amount of a TYK2 inhibitor, wherein the elevated SIGLEC1 is elevated as compared to a patient not having, or having mild, hidradenitis suppurativa, thereby upon administration reducing the expression of SIGLEC1 in the patient.

In addition, provided herein is a method of treating a subject suffering from hidradenitis suppurativa, comprising: providing a biological sample from the subject; assaying gene expression of one or more IFN-1 genes in the biological sample; determining the expression value of the IFN-1 gene(s); and if the expression value is above a threshold value, administering a TYK2 inhibitor to the patient.

The present disclosure also provides for a method of treating a patient suffering from moderate or severe hidradenitis suppurativa comprising administering to the patient an effective amount of a compound represented by:

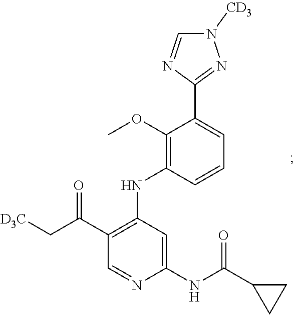

or a pharmaceutically acceptable salt thereof, wherein after at least 16 weeks of daily or twice daily administration, wherein the patient has at least a 50% reduction in the total abscess and inflammatory nodule (AN) count with no increase in abscess and no increase in draining fistula count compared with baseline.

### BRIEF DESCRIPTION OF THE DRAWINGS

FIG. 1 depicts a median IFN signature violin plot of median IFN gene expression in disease (HS) and control states.

FIG. 2 depicts a median IFN signature density plot of median IFN gene expression in disease (HS) and control states.

FIG. 3 depicts a density plot of SIGLEC1 gene expression in disease (HS) and control states.

FIG. 4 shows dose-dependent median IFN gene expression levels over time for HERC5, IFI27, IFIT1, RSAD2 in healthy volunteers after 14 days Compound A versus placebo.

FIG. 5 shows dose-dependent median IFN gene expression levels (log scale) over time for HERC5, IFI27, IFIT1, RSAD2 in healthy volunteers after 14 days Compound A versus placebo.

FIG. 6 shows dose-dependent median IFN gene expression levels over time for SIGLEC1 in healthy volunteers after 14 days Compound A versus placebo.

FIG. 7 shows dose-dependent median IFN gene expression levels (log scale) over time for SIGLEC1 in healthy volunteers after 14 days Compound A versus placebo.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

As used herein and in the appended claims, the singular forms “a,” “an,” and “the” include plural referents unless the context clearly dictates otherwise. Thus, for example, reference to “an agent” includes a plurality of such agents, and equivalents thereof known to those skilled in the art, and so forth. When ranges are used herein, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease. Also, for purposes herein, “treatment,” “prevention,” “amelioration,” or “inhibition” encompass delaying the onset of the disorder, or a symptom or condition thereof.

The terms “effective amount” or “therapeutically effective amount,” as used herein, refer to a sufficient amount of a compound or composition disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

### Methods

Disclosed herein, for example, is a method of treating a patient suffering from moderate or severe hidradenitis suppurativa, and in need of treatment, comprising administering to the patient an effective amount of a TYK2 inhibitor. In some embodiments, a hidradenitis suppurativa lesional skin sample from the patient before administration of the TYK2 inhibitor has elevated Type I IFN-induced sialic acid-binding immunoglobulin-like lectin1 (SIGLEC1) expression. In further embodiments, the elevated Type I IFN induced sialic acid-binding immunoglobulin-like lectin1 (SIGLEC1) expression is elevated as compared to, e.g, the SIGLEC1 expression in the whole blood of the patient, or as compared to a patient not suffering from moderate to severe hidradenitis suppurativa. For example, in some embodiments a lesional skin sample from the patient before administration of the TYK2 inhibitor has increased IFN-1 gene expression. In certain embodiments, the increased IFN-1 gene expression is one or more of increased gene expression of: OAS3, OAS2, CD8A, EPSTI1, BST2, RNF213, EIF2AK2, IFIT2, IFIT3, SP100, SP110, LY6E, MX1, IFI6, RTP4, XAF1, PATL2, STAT1, and SIGLEC1.

Also described herein is a method of treating moderate or severe hidradenitis suppurativa in a patient having elevated Type I IFN-induced sialic acid-binding immunoglobulin-like lectin1 (SIGLEC1) expression, and in need of treatment, comprising administering to the patient an effective amount of a TYK2 inhibitor, and thereby upon administration reducing the expression of SIGLEC1 in the patient.

Further disclosed herein, for example, is a method of treating a patient having elevated sialic acid-binding immunoglobulin-like lectin1 (SIGLEC1) expression (e.g., expression in blood and/or skin) and suffering from moderate to severe hidradenitis suppurativa, comprising: administering to the patient an effective amount of a TYK2 inhibitor, wherein the elevated SIGLEC1 is elevated as compared to a patient not having, or having mild, hidradenitis suppurativa, thereby upon administration reducing the expression of SIGLEC1 in the patient.

In addition, disclosed herein is a method of treating a subject suffering from hidradenitis suppurativa, comprising: providing a biological sample from the subject; assaying gene expression of one or more IFN-1 genes in the biological sample; determining the expression value of the IFN-1 gene(s); and if the expression value is above a threshold value, administering a TYK2 inhibitor to the patient. In certain embodiments, the IFN-1 gene is one or more of: OAS3, OAS2, CD8A, EPSTI1, BST2, RNF213, EIF2AK2, IFIT2, IFI6, and SIGLEC1.

In some embodiments, the TYK2 inhibitor is selected from the group consisting of, for example, GLPG3121, GLPG3667, VTX-958, ICP-332, BGB-23339,

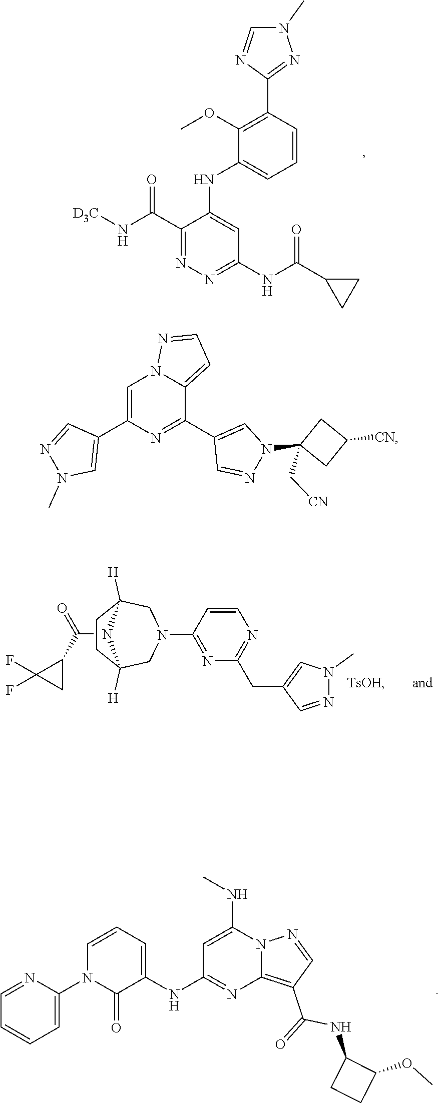

In other embodiments, a disclosed TYK2 inhibitor contemplated herein may be represented by, for example:

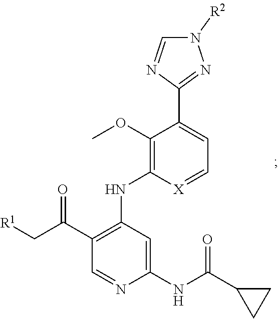

or a pharmaceutically acceptable salt thereof; wherein X is CH or N; and R¹ and R² are independently for each occurrence selected from CH₃ and CD₃.

For example, in some embodiments a disclosed TYK2 inhibitor may be selected from the group consisting of, e.g.,

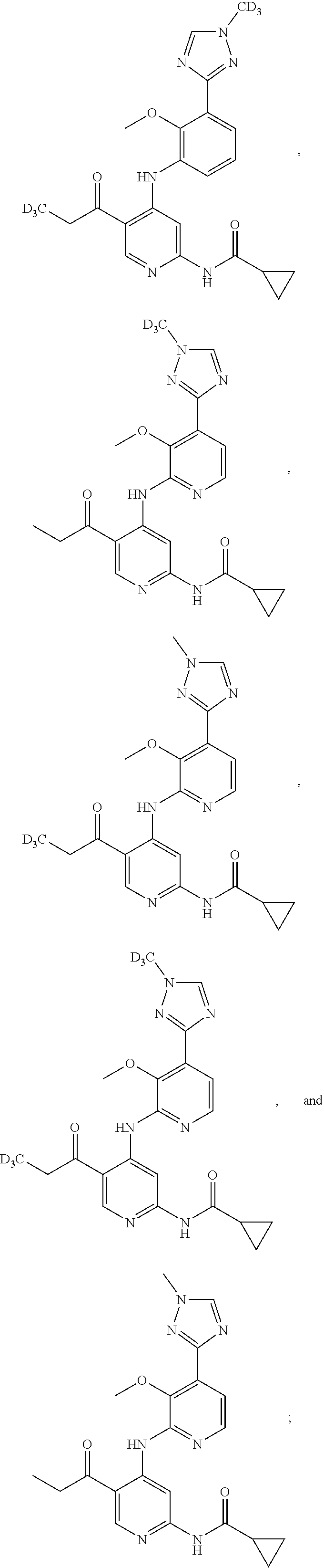

or a pharmaceutically acceptable salt thereof.

In some embodiments, the patient has hidradenitis suppurativa lesions in at least 2 distinct anatomic areas before administration of the Tyk2 inhibitor. In other embodiments, the patient has a hidradenitis suppurativa lesion at Hurley Stage II or III and/or the patient has a total abscess and inflammatory (AN) count ≥5 before administration of the Tyk2 inhibitor. In certain embodiments, the patient has had an inadequate response to at least a 3-month course of oral antibiotics for treatment of hidradenitis suppurativa or has exhibited recurrence after discontinuation of, or demonstrated intolerance to, or have a contraindication to oral antibiotics for treatment of hidradenitis suppurativa before the administration of the Tyk2 inhibitor.

The present disclosure also provides for a method of treating a patient suffering from moderate or severe hidradenitis suppurativa, and in need of treatment, comprising administering to the patient an effective amount of a compound represented by:

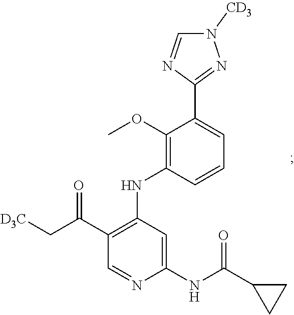

or a pharmaceutically acceptable salt thereof, wherein after at least 16 weeks of daily or twice daily administration, wherein the patient has at least a 50% reduction in the total abscess and inflammatory nodule (AN) count with no increase in abscess and no increase in draining fistula count compared with baseline.

In some embodiments, the patient has at least a 75%, 90% or 100% reduction in the total abscess and inflammatory nodule (AN) count with no increase in abscess and no increase in draining fistula count compared with baseline after 16 weeks or more of daily or twice daily administration. In other embodiments, the patient achieves a HS-PGA of patients achieving HS-PGA of clear, minimal, or mild with at least a 2-grade improvement after 16 weeks or more of daily or twice daily administration. In still other embodiments, the patient achieves at least a 30% reduction from baseline in Patient's Global Assessment of Skin Pain after 16 weeks or more of daily or twice daily administration, where before the administration, the patient had a NRS ≥3. In certain embodiments, the patient achieves AN count of 0, 1, or 2 after 16 weeks or more of daily or twice daily administration.

In some embodiments, the methods described herein comprise orally administering or topically administering the compound to the patient, for example, orally administering 10 mg to 80 mg of the compound once or twice daily; for example, orally administering 40 mg or 60 mg of the compound once or twice daily. In other embodiments, the methods described herein further comprise administering an antibiotic to the patient.

In certain embodiments, the compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration (e.g., oral administration) and standard pharmaceutical practice as described, for example, in Remington: The Science and Practice of Pharmacy (Gennaro, 21^{st} Ed. Mack Pub. Co., Easton, PA (2005)).

In certain embodiments, the compound provided herein is substantially pure, in that it contains less than about 5%, or less than about 1%, or less than about 0.1%, of other organic small molecules, such as unreacted intermediates or synthesis by-products that are created, for example, in one or more of the steps of a synthesis method.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). Optimal doses are generally determined using experimental models and/or clinical trials. The optimal dose depends upon the body mass, weight, or blood volume of the patient.

For example, in some embodiments a disclosed compound or composition may be administered once daily to the patient. In other embodiments, a disclosed compound or composition may be administered twice daily to the patient.

In some embodiments, a disclosed compound or pharmaceutical composition is formulated for oral administration. In some embodiments, the pharmaceutical composition is formulated as a tablet, a pill, a capsule, a liquid, a suspension, a dispersion, a solution, or an emulsion. In some embodiments, the pharmaceutical composition is formulated as a tablet, for example, a tablet formulated for oral administration.

Suitable doses and dosage regimens are determined by conventional range-finding techniques known to those of ordinary skill in the art. Generally, treatment is initiated with smaller dosages that are less than the optimum dose of the compound disclosed herein. Thereafter, the dosage is increased by small increments until the optimum effect under the circumstances is reached. In some embodiments, the present method involve the administration of about 0.1 μg to about 50 mg of at least one compound described herein per kg body weight of the subject. For a 70 kg patient, dosages of from about 10 μg to about 200 mg of the compound disclosed herein would be more commonly used, depending on a subject's physiological response.

By way of example only, the dose of the compound described herein for methods of treating a disease as described herein is about 0.001 to about 1 mg/kg body weight of the subject per day, for example, about 0.001 mg, about 0.002 mg, about 0.005 mg, about 0.010 mg, 0.015 mg, about 0.020 mg, about 0.025 mg, about 0.050 mg, about 0.075 mg, about 0.1 mg, about 0.15 mg, about 0.2 mg, about 0.25 mg, about 0.5 mg, about 0.75 mg, or about 1 mg/kg body weight per day. In some embodiments, the dose of compound described herein for the described methods is about 1 to about 1000 mg/kg body weight of the subject being treated per day, for example, about 1 mg, about 2 mg, about 5 mg, about 10 mg, about 15 mg, about 20 mg, about 25 mg, about 50 mg, about 75 mg, about 100 mg, about 150 mg, about 200 mg, about 250 mg, about 500 mg, about 750 mg, or about 1000 mg per day.

Further, by way of example only, the dose of the compound for methods of treating a disease as described herein may be a fixed dose, e.g., a fixed dose of 10 mg, 15 mg, 20 mg, 25 mg, 30 mg, 35 mg, 40 mg, 45 mg, 50 mg, 55 mg, 60 mg, 65 mg, 70 mg, 75 mg, or 80 mg.

In certain instances, a compound described herein is administered in combination with a second therapeutic agent, for example, an antibiotic. In some embodiments, the benefit experienced by a patient is increased by administering a compound described herein with a second therapeutic agent that also has therapeutic benefit. In one specific embodiment, a compound described herein is co-administered with a second therapeutic agent, e.g., an antibiotic, wherein the compound described herein and the second therapeutic agent modulate different aspects of the disease, disorder or condition being treated, thereby providing a greater overall benefit than administration of either therapeutic agent alone. The overall benefit experienced by the patient is simply additive of the two therapeutic agents or the patient experiences a synergistic benefit.

It is understood that the dosage regimen to treat, prevent, or ameliorate the condition(s) for which relief is sought, is modified in accordance with a variety of factors (e.g., the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

The compounds describe herein are administered before, during, or after the occurrence of a disease or condition, and the timing of administering the composition containing a compound varies. Thus, in one embodiment, the compounds and compositions described herein are used as a prophylactic and are administered continuously to subjects with a propensity to develop conditions or diseases in order to prevent the occurrence of the disease or condition. In another embodiment, the compounds are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

The following examples are provided for illustrative purposes only, and are not intended to limit the scope of the disclosure.

### Example 1: Differential Gene Expression (DGE) Analysis in a Case Control HS Dataset

### Analytical Methods

A differential gene expression (DGE) analysis in a case control HS dataset was conducted. The dataset used in the analysis was imported from NCBI's BioProjects under accession number PRJNA647407. The dataset was contributed by “Prens E P, Gudjonsson J E, Tsoi LC” for hidradenitis suppurativa. The dataset was a case control set consisting of transcriptome profiling in lesional tissue as well as whole blood. The analytical dataset was raw counts at the gene level. Custom quantification was performed using STAR->Salmon->RSEM. Differential expression analysis was carried using a DESeq2 program for normalization, visualization, and analysis of RNA-seq data, comparing cases to controls. A geometric mean was calculated for each gene across all samples. The counts for a gene in each sample was then divided by this mean, with the median of these ratios in a sample being the size factor for that sample. Genes were removed that contained less than 20 counts summed across all individuals.

### Results

Differential gene expression analysis was also conducted on interferon type-1 (IFN-1) cytokine genes. Table 1 shows the top differentially expressed IFN-1 genes in lesional tissue and whole blood samples obtained from patients with hidradenitis suppurativa. As shown in Table 1, Type I IFN induced sialic acid-binding immunoglobulin-like lectin1 (SIGLEC1) expression is elevated as compared to the SIGLEC1 expression in patients having hidradenitis suppurativa as compared to base levels.

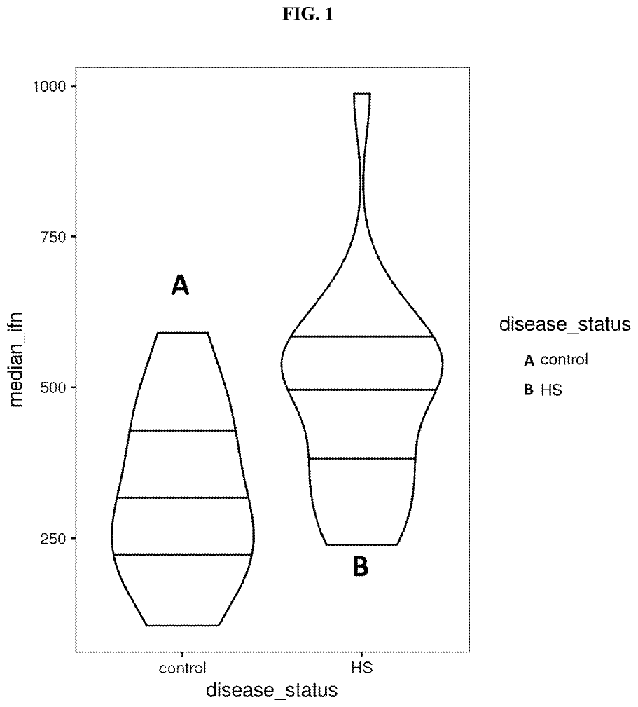

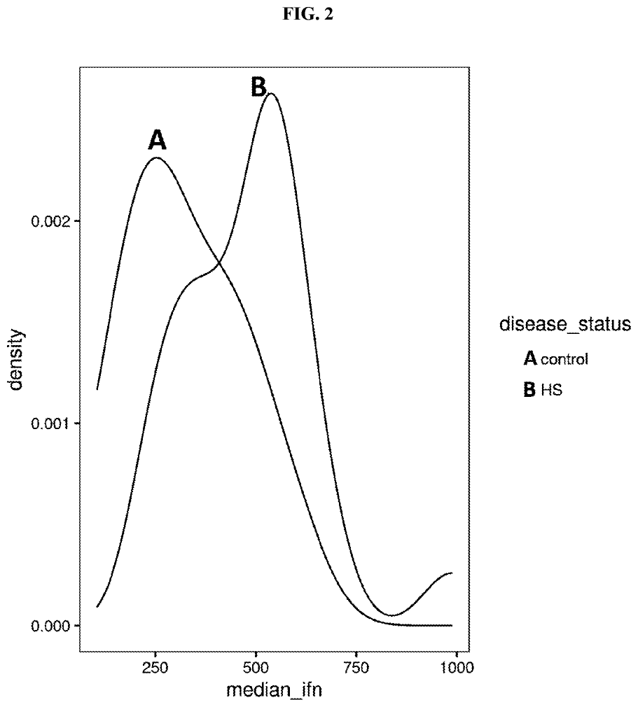

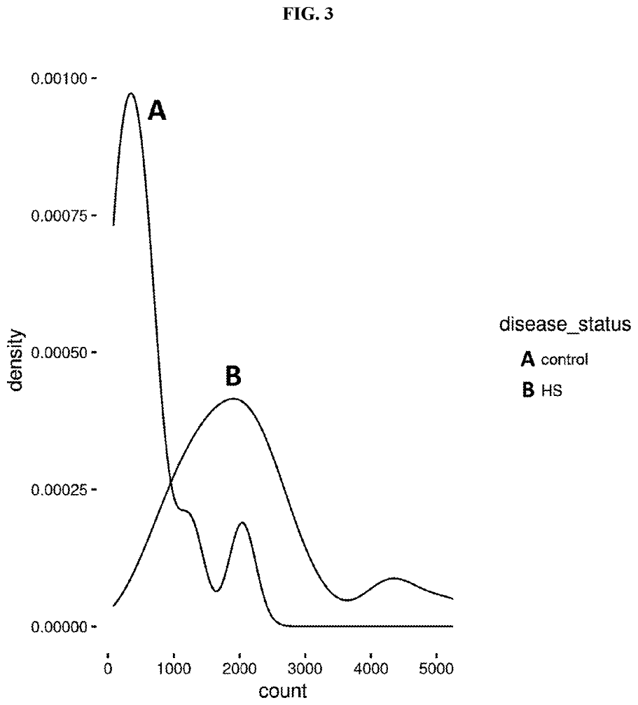

FIG. 1 depicts a median IFN signature violin plot of median IFN gene expression in disease and control states. As shown if FIG. 1, subjects having hidradenitis suppurativa displayed increased IFN gene expression (shown as median) compared to non-disease status. FIG. 2 depicts a median IFN signature density plot of median IFN gene expression in disease and control states. Similar to FIG. 1, FIG. 2 also indicates that subjects having hidradenitis suppurativa displayed an increase in IFN expression (shown as median) compared to non-disease status. FIG. 3 depicts a density plot of SIGLEC1 gene expression in disease and control states. As shown in FIG. 3, an increase in median SIGLEC1 expression was observed in subjects hidradenitis suppurativa relative to non-disease state.

TABLE 1

| Gene symbol | baseMean | log2FoldChange | lfcSE | stat | pvalue | padj |
|---|---|---|---|---|---|---|
| CD8A | 559.5 | 2.027 | 0.315 | 6.434 | 1.24e−10 | 3.103e−09 |
| OAS2 | 1616 | 1.732 | 0.2692 | 6.434 | 1.244e−10 | 3.108e−09 |
| BST2 | 1332 | 1.654 | 0.2576 | 6.419 | 1.37e−10 | 3.38e−09 |
| EPSTI1 | 824.7 | 2.063 | 0.3216 | 6.413 | 1.427e−10 | 3.505e−09 |
| ISG15 | 539.4 | 1.526 | 0.2387 | 6.391 | 1.65e−10 | 3.987e−09 |
| RNF213 | 8932 | 1.041 | 0.1737 | 5.992 | 2.068e−09 | 3.785e−08 |
| IFI6 | 1487 | 1.224 | 0.222 | 5.513 | 3.535e−08 | 4.843e−07 |
| SIGLEC1 | 1696 | 1.8 | 0.3586 | 5.019 | 5.195e−07 | 5.356e−06 |
| IFIT2 | 614.2 | 1.123 | 0.2377 | 4.726 | 2.286e−06 | 1.95e−05 |
| EIF2AK2 | 1963 | 0.559 | 0.121 | 4.62 | 3.833e−06 | 3.082e−05 |
| OAS3 | 2499 | 1.031 | 0.228 | 4.52 | 6.183e−06 | 4.714e−05 |
| SP100 | 2608 | 0.4803 | 0.109 | 4.407 | 1.048e−05 | 7.493e−05 |
| RTP4 | 172.1 | 1.15 | 0.2673 | 4.301 | 1.703e−05 | 0.0001157 |
| LY6E | 5757 | −0.7916 | 0.189 | −4.188 | 2.813e−05 | 0.0001796 |
| SP110 | 1169 | 0.6296 | 0.1538 | 4.094 | 4.239e−05 | 0.0002569 |
| XAF1 | 1654 | 0.9979 | 0.2554 | 3.907 | 9.336e−05 | 0.0005101 |
| MX1 | 1688 | 0.9689 | 0.2548 | 3.803 | 0.0001432 | 0.0007427 |
| STAT1 | 8279 | 1.073 | 0.2847 | 3.769 | 0.0001641 | 0.0008358 |
| PATL2 | 67.6 | 1.042 | 0.2894 | 3.599 | 0.0003196 | 0.001482 |
| IFIT3 | 1327 | 0.926 | 0.2773 | 3.339 | 0.0008398 | 0.0034 |
| IFI44 | 500.9 | 0.8013 | 0.2739 | 2.926 | 0.003433 | 0.01129 |
| IFI27 | 4515 | 0.8449 | 0.3088 | 2.736 | 0.006216 | 0.01848 |
| LAG3 | 156.4 | 0.9958 | 0.367 | 2.713 | 0.006667 | 0.01964 |
| SPATS2L | 2808 | 0.2462 | 0.09463 | 2.602 | 0.009279 | 0.02596 |
| RSAD2 | 286.7 | 0.7343 | 0.2988 | 2.458 | 0.01398 | 0.03627 |
| HERC5 | 264.5 | 0.4479 | 0.1866 | 2.4 | 0.01639 | 0.04144 |
| CXCL10 | 684.3 | 1.789 | 0.7689 | 2.326 | 0.02001 | 0.04899 |
| LAMP3 | 1275 | 0.6226 | 0.3156 | 1.973 | 0.04853 | 0.1012 |
| IFIT1 | 543 | 0.5069 | 0.2615 | 1.938 | 0.05262 | 0.1082 |
| OAS1 | 1326 | 0.3801 | 0.1992 | 1.908 | 0.05643 | 0.1145 |
| IFI44L | 489.1 | 0.5026 | 0.3174 | 1.583 | 0.1133 | 0.1992 |
| HBB | 1727 | −0.5802 | 0.5911 | −0.9815 | 0.3263 | 0.4558 |
| ISG20 | 678.2 | 0.2456 | 0.32 | 0.7673 | 0.4429 | 0.5708 |
| RGS1 | 946.2 | −0.2499 | 0.5435 | −0.4599 | 0.6456 | 0.7495 |
| PLSCR1 | 1827 | −0.1 | 0.2692 | −0.3716 | 0.7102 | 0.7989 |
| USP18 | 218.4 | 0.02691 | 0.1653 | 0.1628 | 0.8706 | 0.9144 |
| IFI16 | 9461 | 0.02429 | 0.1952 | 0.1244 | 0.901 | 0.9361 |

### Example 2: Study Description

A randomized, double-blind, placebo-controlled study to assess the safety, efficacy, and pharmacokinetics of multiple dose levels of Compound A in adult patients with moderate to severe hidradenitis suppurativa is conducted. N-(4-((2-Methoxy-3-(1-(methyl-d₃)-1H-1,2,4-triazol-3-yl)phenyl)amino)-5-(propanoyl-3,3,3-d₃)pyridin-2-yl)cyclopropanecarboxamide (Compound A) can be prepared according to the synthetic procedures described in WO 2020/086616, the entire content of which is incorporated by reference herein.

Primary Objectives: To compare the hidradenitis suppurativa clinical response (HiSCR) between doses of Compound A and placebo after 16 weeks of treatment.

Primary Endpoints: Proportion of patients achieving HiSCR (defined as at least a 50% reduction in the total abscess and inflammatory nodule (AN) count with no increase in abscess and no increase in draining fistula count compared with baseline) at week 16 between doses of Compound A and placebo.

Secondary Objectives: To compare Hidradenitis Suppurativa Clinical Response (HiSCR75, HiSCR90, and HiSCR100) from baseline to week 16; to compare the change from baseline in Hidradenitis Suppurativa Physician's Global Assessment (HS-PGA) 6-point score at week 16; to compare the change in baseline lesion count between doses of Compound A and placebo; to compare Patient's Global Assessment of Skin Pain among patients with at least 30% reduction from baseline (numeric rating scale 30 [NRS30]) between doses of Compound A and placebo; to compare International Hidradenitis Suppurativa Severity Scoring System (IHS4) score over time; to assess the change from baseline in Dermatology Life Quality Index (DLQI) at week 16; to characterize the pharmacokinetics (PK) of multiple dose levels of Compound A; to assess the safety and tolerability of multiple dose levels of Compound A.

Secondary Endpoints: Proportion of patients achieving 75% (HiSCR75), 90% (HiSCR90), and 100% (HiSCR100) improvements from baseline AN count at week 16; proportion of patients achieving HS-PGA of clear, minimal, or mild with at least a 2-grade improvement relative to baseline at week 16; change and percentage change from baseline in lesion count (abscess count, inflammatory nodule count, abscess and inflammatory nodule count, and draining fistula count) over time; proportion of patients achieving at least a 30% reduction from baseline in Patient's Global Assessment of Skin Pain at W\week 16 (among patients with baseline NRS ≥3); change from baseline in IHS4 score over time; change from baseline in DLQI at week 16 in Compound A compared with placebo; plasma PK parameters of Compound A; incidence of treatment-emergent adverse events and serious adverse events (SAEs).

Exploratory Objectives: To compare AN count of 0, 1, or 2 at week 16; to compare AN count of 0, 1, or 2 over time through week 16 by Hurley stage at baseline; to compare draining fistula count over time through week 16; to assess the change in erythema over time; to assess the change in European Quality of Life 5-Dimensions 5-Levels Health Questionnaire (EQ-5D-5L); to assess the change in Hidradenitis Suppurativa Quality of Life (HISQOL); to assess the change in hidradenitis suppurativa (HS) and tyrosine kinase 2 (TYK2)-related skin-based biomarkers with Compound A treatment; to assess the change in HS and TYK2-related blood-based biomarkers with Compound A treatment; to assess the change in transcriptomic- and proteomic-based biomarkers in blood and skin with Compound A treatment.

Exploratory Endpoints: Proportion of patients achieving AN count of 0, 1, or 2 at week 16; proportion of patients achieving AN count of 0, 1, or 2 over time through week 16 by Hurley stage at baseline; change from baseline in the draining fistula count over time, defined as the mean change from baseline in the draining fistulas count at each visit; change in baseline erythema assessment over time; change from baseline in EQ-5D-5L at week 16 in Compound A compared with placebo; change from baseline in HISQOL at week 16 in Compound A compared with placebo; change from baseline in HS and TYK2-related skin biomarkers in response to Compound A treatment; change from baseline in HS and TYK2-related blood-based biomarkers in response to Compound A treatment; change from baseline in transcriptomic and proteomic expression in response to Compound A treatment.

### Study Design:

This is a randomized, double-blind, placebo-controlled study in adult patients with moderate to severe HS. The purpose of this study is to assess the clinical efficacy, safety, PK, and pharmacodynamics (PD) of multiple dose levels of Compound A compared with placebo in adult patients with moderate to severe HS. The overall study duration from screening through the double-blind treatment period will be up to 20 weeks for patients participating in the open-label extension study and 24 weeks for patients not participating in the open-label extension study. The double-blind treatment period will be 16 weeks. Patients who complete the 16-week double-blind treatment period will be offered to enroll into a separate open-label extension study. Patients who do not consent to enroll into the open-label extension study will participate in an additional 4-week posttreatment safety follow-up period.

Patients who meet all eligibility criteria will be randomly assigned in a 1:1:1:1 ratio to receive oral doses of Compound A at 1 of 3 dose levels (40 mg once daily [QD], 60 mg QD, or 40 mg twice daily [BID]) or placebo. Approximately 260 patients are planned to be enrolled into the study (4 treatment groups with approximately 65 patients per treatment group).

Efficacy assessments include disease activity skin assessments (HiSCR, HS-PGA, IHS4, AN count, erythema assessment, lesion count, and Hurley staging) and patient-reported outcome assessments (DLQI, EQ-5D-5L, and Patient's Global Assessment of Skin Pain [NRS], and HISQOL). Exploratory PD and biomarker assessments include both blood and skin-based transcriptomic, proteomic, and other TYK2 relevant and disease relevant markers.

### Example 3: Gene Expression Analysis in Healthy Volunteers

A study was conducted to assess IFN1 gene expression in healthy volunteers administered a regimen of Compound A. The data set consists of 48 subjects with transcriptome-wide RNA-sequencing data collected for each subject per time-point. For each subject, time points included a pre-dose at day 1, a pre-dose at day 14, and a 4 hour post-day 14 dose. A Wald test was used to assess the association of the various doses at post-dose day 14 versus placebo. To assess potentially off target effects, a secondary transcriptome-wide analysis was performed. Multiple test correction was completed using a Benjamini-Hochberg cutoff of 0.05 within the primary and secondary analyses separately.

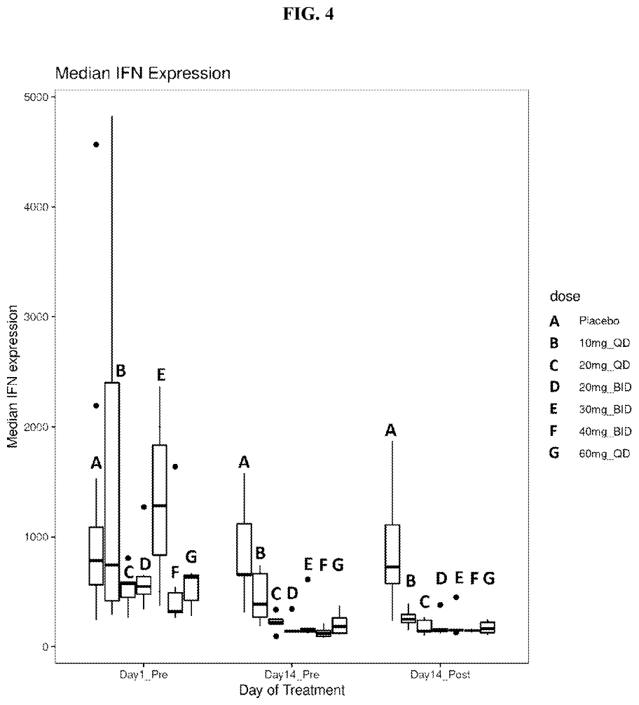

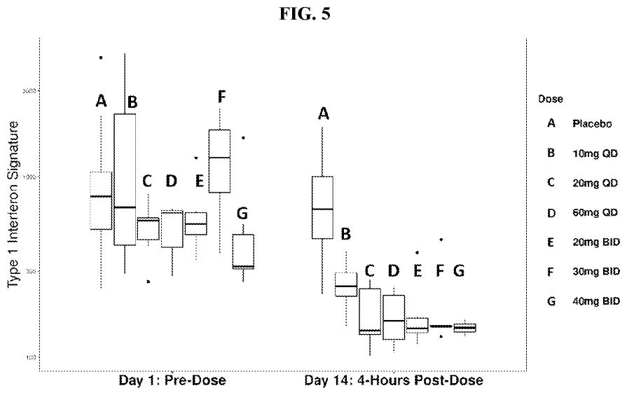

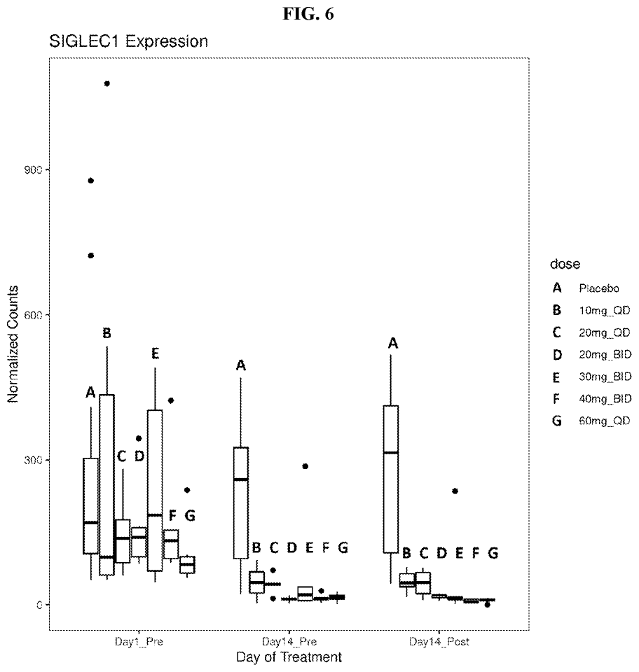

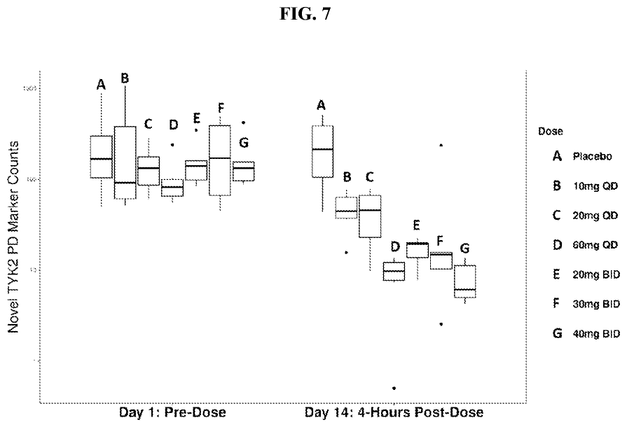

For example FIG. 4 and FIG. 5 show dose-dependent median IFN gene expression levels over time for HERC5, IFI27, IFIT1, RSAD2. As shown in FIG. 4 and FIG. 5, a substantial decrease in IFN expression was observed after 14 days post-treatment with Compound A. FIG. 6 and FIG. 7 shows dose-dependent median IFN gene expression levels over time for SIGLEC1. As shown in FIG. 6 and FIG. 7, SIGLEC1 was decreased after a 14-day regimen of treatment with Compound A.

Table 2, for example, shows IFN gene set results after 14 days of treatment with Compound A versus placebo (P-value based on Wald test). As shown in Table 2, a significant association of Compound A with SIGLEC1 gene expression was observed.

TABLE 2

| Gene | | Mean Base | | BH-Adjusted |
|---|---|---|---|---|
| Symbol | log2FoldChange | Expression | P-value | P-value |
| SIGLEC1 | −3.579 | 124.3 | 3.202e−05 | 0.0004803 |
| MX1 | −1.438 | 2220 | 0.002859 | 0.02144 |
| IFIT1 | −1.676 | 610.3 | 0.01656 | 0.08278 |
| CXCL10 | −1.589 | 12.51 | 0.07494 | 0.281 |
| SPATS2L | −0.5117 | 113.5 | 0.1219 | 0.3318 |
| IFI44L | −1.043 | 1509 | 0.1459 | 0.3318 |
| OAS3 | −0.7134 | 2568 | 0.1549 | 0.3318 |
| ISG15 | −0.8721 | 105.4 | 0.2001 | 0.3496 |
| IFI27 | −1.082 | 33.67 | 0.2098 | 0.3496 |
| IFI44 | −0.3773 | 360.5 | 0.4341 | 0.6512 |
| RSAD2 | −0.4713 | 823.1 | 0.4992 | 0.6807 |
| EPSTI1 | −0.2266 | 579.7 | 0.5786 | 0.7188 |
| ISG20 | 0.08851 | 3376 | 0.623 | 0.7188 |
| OAS1 | −0.1127 | 2267 | 0.7548 | 0.8087 |
| OAS2 | −0.008935 | 6314 | 0.9763 | 0.9763 |

Table 3 shows changes in IFN gene expression in healthy volunteers after 7 days treatment with 40 mg of compound A, twice daily, versus placebo. As shown in Table 3, SIGLEC1 was observed to be the top differentially expressed gene in treated versus placebo.

TABLE 3

| gene_symbol | log2FoldChange | baseMean | pvalue |
|---|---|---|---|
| SIGLEC1 | −3.626 | 148 | 0.0009462 |
| SYN2 | 2.239 | 13.89 | 0.0009678 |
| CETP | 1.503 | 30.81 | 0.00539 |
| SOCS3 | 1.421 | 1032 | 0.007957 |
| HJURP | 1.007 | 40.18 | 0.01959 |
| CHRM3 | −1.521 | 15.87 | 0.02508 |
| OSM | 1.157 | 208.5 | 0.02553 |

Table 4 shows results of a secondary analysis to assess potentially off target effects (contrasting day 14; 4 hour post-treatment versus placebo). As shown in Table 4, no significant off target associations were observed after multiple test corrections.

TABLE 4

| Gene | | Mean Base | | BH-Adjusted |
|---|---|---|---|---|
| Symbol | log2FoldChange | Expression | P-value | P-value |
| SIGLEC1 | −3.579 | 124.3 | 2.651e−05 | 0.5262 |
| IFIT1 | −1.676 | 610.3 | 0.002868 | 1 |
| MX1 | −1.438 | 2220 | 0.004562 | 1 |
| CDNF | 1.185 | 18.41 | 0.004677 | 1 |
| LRP1 | −1.314 | 2334 | 0.005593 | 1 |
| IFI6 | −1.206 | 306.2 | 0.01414 | 1 |
| JPH4 | −1.043 | 23.98 | 0.03419 | 1 |
| LRRC75B | −1.205 | 10.1 | 0.03579 | 1 |
| ENSG00000288643 | −1.541 | 29.29 | 0.03931 | 1 |

### Example 4: SIGLEC1 Expression in Patients with Skin Disorders vs. Healthy Subjects

Publicly available gene expression data sets were analyzed to test for statistically significant upregulation of SIGLEC1 in patients with skin disorders versus healthy subjects. The results are shown in Table 5. As shown in Table 5, the changes in SIGLEC1 were significant, with p-value adjusted for multiple testing <0.1 and enriched in patients with skin disorders compared to healthy subjects.

TABLE 5

| Disease/Disorder | Sample | log2FoldChange | padj |
|---|---|---|---|
| systemic lupus erythematosus | blood | 3.150892461 | 4.49E−22 |
| cutaneous lupus erythematous | skin | 2.157144433 | 3.99E−06 |
| hidradenitis suppurativa skin | skin | 1.783698872 | 5.96E−06 |
| subacute cutaneous lupus erythematosus | skin | 2.197338045 | 0.001234863 |
| dermatomyositis | muscle | 1.855137889 | 0.002381401 |
| juvenile dermatomyositis | blood | 2.903796856 | 0.038502825 |
| chronic cutaneous lupus erythematous | skin | 2.028622672 | 0.038829559 |
| acute cutaneous lupus erythematous | skin | 2.198402607 | 0.0545622 |
| rheumatoid arthritis | blood | 0.317236946 | 0.091346071 |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating a patient suffering from moderate or severe hidradenitis suppurativa comprising administering to the patient an effective amount of a TYK2 inhibitor.

2. The method of claim 1, wherein a hidradenitis suppurativa lesional skin sample from the patient before administration of the TYK2 inhibitor has elevated Type I IFN-induced sialic acid-binding immunoglobulin-like lectin1 (SIGLEC1) expression.

3. The method of claim 2, wherein the elevated Type I IFN induced sialic acid-binding immunoglobulin-like lectin1 (SIGLEC1) expression is elevated as compared to the SIGLEC1 expression in the whole blood of the patient, or as compared to a patient not suffering from moderate to severe hidradenitis suppurativa.

4. The method of claim 1, wherein a lesional skin sample from the patient before administration of the TYK2 inhibitor has increased IFN-1 gene expression.

5. The method of claim 4, wherein the increased IFN-1 gene expression is one or more of increased gene expression of: OAS3, OAS2, CD8A, EPSTI1, BST2, RNF213, EIF2AK2, IFIT2, IFIT3, SP100, SP110, LY6E, MX1, IFI6, RTP4, XAF1, PATL2, STAT1, and SIGLEC1.

6. A method of treating moderate or severe hidradenitis suppurativa in a patient having elevated Type I IFN-induced sialic acid-binding immunoglobulin-like lectin1 (SIGLEC1) expression, and in need of treatment, comprising administering to the patient an effective amount of a TYK2 inhibitor, and thereby upon administration reducing the expression of SIGLEC1 in the patient.

7. A method of treating a patient having elevated sialic acid-binding immunoglobulin-like lectin1 (SIGLEC1) expression and suffering from moderate to severe hidradenitis suppurativa, comprising:
administering to the patient an effective amount of a TYK2 inhibitor, wherein the elevated SIGLEC1 is elevated as compared to a patient not having, or having mild, hidradenitis suppurativa, and
thereby upon administration reducing the expression of SIGLEC1 in the patient.

8. A method of treating a subject suffering from hidradenitis suppurativa, comprising:
providing a biological sample from the subject;
assaying gene expression of one or more IFN-1 genes in the biological sample;
determining the expression value of the IFN-1 gene(s); and
if the expression value is above a threshold value, administering a TYK2 inhibitor to the patient.

9. The method of claim 8, wherein the IFN-1 gene is one or more of: OAS3, OAS2, CD8A, EPSTI1, BST2, RNF213, EIF2AK2, IFIT2, IFI6, and SIGLEC1.

10. The method of claim 8 or 9, wherein the biological sample is the subject's blood or tissue.

11. The method of any one of claims 1-10, wherein the TYK2 inhibitor is selected from the group consisting of GLPG3121, GLPG3667, VTX-958, ICP-332, BGB-23339,

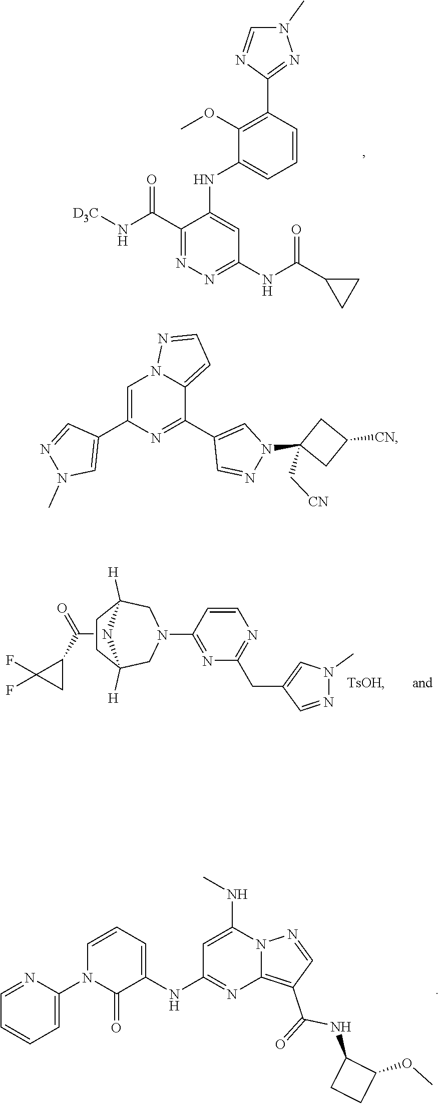

12. The method of any one of claims 1-10, wherein the TYK2 inhibitor is represented by: 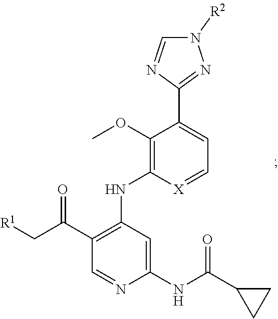
or a pharmaceutically acceptable salt thereof;
wherein:
X is CH or N; and
R¹ and R² are independently for each occurrence selected from CH₃ and CD₃.

13. The method of any one of claims 1-12, wherein the patient has hidradenitis suppurativa lesions in at least 2 distinct anatomic areas before administration of the TYK2 inhibitor.

14. The method of any one of claims 1-13, wherein the patient has a hidradenitis suppurativa lesion at Hurley Stage II or III and/or the patient has a total abscess and inflammatory (AN) count ≥5 before administration of the TYK2 inhibitor.

15. The method of any one of claims 1-14, wherein the patient has had an inadequate response to at least a 3-month course of oral antibiotics for treatment of hidradenitis suppurativa or has exhibited recurrence after discontinuation of, or demonstrated intolerance to, or have a contraindication to oral antibiotics for treatment of hidradenitis suppurativa before the administration of the TYK2 inhibitor.

16. A method of treating a patient suffering from moderate or severe hidradenitis suppurativa comprising administering an effective amount of a compound represented by: 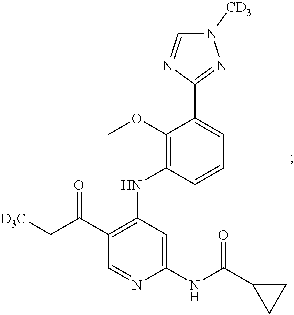
or a pharmaceutically acceptable salt thereof;
wherein after at least 16 weeks of daily or twice daily administration, wherein the patient has at least a 50% reduction in the total abscess and inflammatory nodule (AN) count with no increase in abscess and no increase in draining fistula count compared with baseline.

17. The method of claim 16, wherein the patient has at least a 75%, 90% or 100% reduction in the total abscess and inflammatory nodule (AN) count with no increase in abscess and no increase in draining fistula count compared with baseline after 16 weeks or more of daily or twice daily administration.

18. The method of claim 16 or 17, wherein the patient achieves a HS-PGA of patients achieving HS-PGA of clear, minimal, or mild with at least a 2-grade improvement after 16 weeks or more of daily or twice daily administration.

19. The method of any one of claims 16-18, wherein the patient achieves at least a 30% reduction from baseline in Patient's Global Assessment of Skin Pain after 16 weeks or more of daily or twice daily administration, where before the administration, the patient had a NRS ≥3.

20. The method of any one of claims 15-18, wherein the patient achieves AN count of 0, 1, or 2 after 16 weeks or more of daily or twice daily administration.

21. The method of any one of claims 15-19, comprising orally administering or topically administering the compound to the patient.

22. The method of any one of claims 15-19, comprising orally administering 10 mg to 80 mg of the compound once or twice daily.

23. The method of any one of claim 21, comprising orally administering 40 mg or 60 mg of the compound once or twice daily.

24. The method of any one of claims 15-22, further comprising administering an antibiotic to the patient.
